SEQUENCE LISTING (15 sequences)

SEQ ID NO: 1 - protein, 416 aa, bovine
MAAGAVVGAWMLVLSLGGTVTGDQNITARIGKPLVLNCKGAPKKPPQQLEWKLNTGRTEAWKVLSPQGDPWDSVARVLPNGSLLLPAVGIQDEGTFRCRATSRSGKETKSNYRVRVYQIPGKPEIVDPASELMAGVPNKVGTCVSEGGYPAGTLNWLLDGKTLIPDGKGVSVKEETKRHPKTGLFTLHSELMVTPARGGALHPTFSCSFTPGLPRRRALHTAPIQLRVWSEHRGGEGPNVDAVPLKEVQLVVEPEGGAVAPGGTVTLTCEAPAQPPPQIHWIKDGRPLPLPPGPMLLLPEVGPEDQGTYSCVATHPSHGPQESRAVSVTIIETGEEGTTAGSVEGPGLETLALTLGILGGLGTVALLIGVIVWHRRRQRKGQERKVPENQEEEEEERAELNQPEEPEAAESSTGGP

SEQ ID NO: 2 - DNA, 1426 bp, bovine
cggagaaggatggcagcaggggcagtggtcggagcctggatgctagtcctcagtctgggggggacagtcacgggggaccaaaacatcacagcccggatcgggaagccactggtgctgaactgcaagggagcccccaagaaaccaccccagcagctggaatggaaactgaacacaggccggacagaagcttggaaagtcctgtctccccagggagacccctgggatagcgtggctcgggtcctccccaacggctccctcctcctgccggctgttgggatccaggatgaggggactttccggtgccgggcaacgagccggagcggaaaggagaccaagtctaactaccgagtccgagtctatcagattcctgggaagccagaaattgttgatcctgcctctgaactcatggctggtgtccccaataaggtggggacatgtgtgtccgaggggggctaccctgcagggactcttaactggctcttggatgggaaaactctgattcctgatggcaaaggagtgtcagtgaaggaagagaccaagagacacccaaagacagggcttttcacgctccattcggagctgatggtgaccccagctcggggaggagctctccaccccaccttctcctgtagcttcacccctggccttccccggcgccgagccctgcacacggcccccatccagctcagggtctggagtgagcaccgaggtggggagggccccaacgtggacgctgtgccactgaaggaagtccagttggtggtagagccagaagggggagcagtagctcctggtggtactgtgaccttgacctgtgaagcccccgcccagcccccacctcaaatccactggatcaaggatggcaggcccctgccccttccccctggccccatgctgctcctcccagaggtagggcctgaggaccagggaacctacagttgtgtggccacccatcccagccatgggccccaggagagccgtgctgtcagcgtcacgatcatcgaaacaggcgaggaggggacgactgcaggctctgtggaagggccggggctggaaaccctagccctgaccctggggatcctgggaggcctggggacagtcgccctgctcattggggtcatcgtgtggcatcgaaggcggcaacgcaaaggacaggagaggaaggtcccggaaaaccaggaggaggaagaggaggagagagcggaactgaaccagccagaggagcccgaggcggcagagagcagcacaggagggccttgaggagcccacggccagacccgatccatcagccccttttcttttcccacactctgttctggccccagaccagttctcctctgtataatctccagcccacatctcccaaactttcttccacaaccagagcctcccacaaaaagtgatgagtaaacacctgccacattta

SEQ ID NO: 3 - protein, 404 aa, Homo sapiens
GAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCRAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 4 - DNA, 1391 bp, Homo sapiens
ggggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgcagggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttgca

SEQ ID NO: 5 - protein, 403 aa, mouse
MPAGTAARAWVLVLALWGAVAGGQNITARIGEPLVLSCKGAPKKPPQQLEWKLNTGRTEAWKVLSPQGGPWDSVAQILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHADEGTYSCVATHPSHGPQESPPVSIRVTETGDEGPAEGSVGESGLGTLALALGILGGLGVVALLVGAILWRKRQPRREERKAPESQEDEEERAELNQSEEAEMPENGAGGP

SEQ ID NO: 6 - DNA, 1348 bp, mouse
gcaccatgccagcggggacagcagctagagcctgggtgctggttcttgctctatggggagctgtagctggtggtcagaacatcacagcccggattggagagccacttgtgctaagctgtaagggggcccctaagaagccgccccagcagctagaatggaaactgaacacaggaagaactgaagcttggaaggtcctctctccccagggaggcccctgggacagcgtggctcaaatcctccccaatggttccctcctccttccagccactggaattgtcgatgaggggacgttccggtgtcgggcaactaacaggcgagggaaggaggtcaagtccaactaccgagtccgagtctaccagattcctgggaagccagaaattgtggatcctgcctctgaactcacagccagtgtccctaataaggtggggacatgtgtgtctgagggaagctaccctgcagggacccttagctggcacttagatgggaaacttctgattcccgatggcaaagaaacactcgtgaaggaagagaccaggagacaccctgagacgggactctttacactgcggtcagagctgacagtgatccccacccaaggaggaaccacccatcctaccttctcctgcagtttcagcctgggccttccccggcgcagacccctgaacacagcccctatccaactccgagtcagggagcctgggcctccagagggcattcagctgttggttgagcctgaaggtggaatagtcgctcctggtgggactgtgaccttgacctgtgccatctctgcccagccccctcctcaggtccactggataaaggatggtgcacccttgcccctggctcccagccctgtgctgctcctccctgaggtggggcacgcggatgagggcacctatagctgcgtggccacccaccctagccacggacctcaggaaagccctcctgtcagcatcagggtcacagaaaccggcgatgaggggccagctgaaggctctgtgggtgagtctgggctgggtacgctagccctggccttggggatcctgggaggcctgggagtagtagccctgctcgtcggggctatcctgtggcgaaaacgacaacccaggcgtgaggagaggaaggccccggaaagccaggaggatgaggaggaacgtgcagagctgaatcagtcagaggaagcggagatgccagagaatggtgccgggggaccgtaagagcacccagatcgagcctgtgtgatggccctagagcagctcccccacattccatcccaattcctccttgaggcacttccttctccaaccagagcccacatgatccatgctgagtaaacatttgatacggc

SEQ ID NO: 7 - protein, 332 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTA

SEQ ID NO: 8 - protein, 22 aa, Homo sapiens
MAAGTAVGAWVLVLSLWGAVVG

SEQ ID NO: 9 - protein, 30 aa, artificial
  Example of amino acid sequence capable of binding to amyloid-beta peptide
AQNITARIGEPCVLKCKGAPKKPPQRLEWK

SEQ ID NO: 10 - protein, 21 aa, artificial
  Forward primer sequence for beta-actin
ACGGCCAGGTCATCACTATTG

SEQ ID NO: 11 - protein, 20 aa, artificial
  Reverse primer sequence for beta-actin
TGGATGCCACAGGATTCCAT

SEQ ID NO: 12 - protein, 35 aa, artificial
  Probe sequence for beta-actin
FAMACGTCTACCAGCGAAGCTACTGCCGTCTAMRA

SEQ ID NO: 13 - protein, 21 aa, artificial
  Forward primer sequence for RAGE
GGATCCCGTCTCAGGGTGTCT

SEQ ID NO: 14 - protein, 21 aa, artificial
  Reverse primer sequence for RAGE
GAGTCCCGTCTCAGGGTGTCT

SEQ ID NO: 15 - protein, 35 aa, artificial
  Probe sequence for RAGE
FAMATTCCCGATGGCAAAGAAACACTCGTGTAMRA

(19) United States
(12) Patent Application Publication
(10) Pub. No.: US 2008/0207499 A1
(43) Pub. Date: Aug. 28, 2008

(54) Rage-related methods for treating and preventing diabetic retinopathy

(76) Inventors: Gaetano Barile, River Vale, NJ (US); Ann Marie Schmidt, Franklin Lakes, NJ (US)

Correspondence Address: COOPER & DUNHAM, LLP, 1185 AVENUE OF THE AMERICAS, NEW YORK, NY, 10036, US

(21) Appl. No.: 11/477,274

(22) Filed: Jun. 28, 2006

Related U.S. Application Data
(60) Provisional application No. 60/695,757, filed on Jun. 29, 2005.

(51) Int. Cl. A61K 38/17; A61P 9/10; A61P 27/02

(52) U.S. Cl. 514/12

(57) ABSTRACT

This invention provides a method for treating diabetic retinopathy in a subject afflicted therewith, comprising administering to the subject's eyes a therapeutically effective amount of soluble RAGE or a derivative thereof, thereby treating diabetic retinopathy in the subject. This invention further provides a method for inhibiting the onset of diabetic retinopathy in a subject afflicted therewith, comprising administering a prophylactically effective amount of soluble RAGE or a derivative thereof to the subject's eyes, thereby inhibiting the onset of diabetic retinopathy in the subject.

This application claims the benefit of U.S. Provisional Application No. 60/695,757, filed Jun. 29, 2005, the contents of which are incorporated herein by reference into the subject application.

Throughout this application, various publications are referred to by Arabic numerals within parentheses. Full citations for these publications are presented immediately before the claims. Disclosures of these publications in their entireties are hereby incorporated by reference into this application in order to more fully describe the state of the art to which this invention pertains.

### BACKGROUND OF THE INVENTION

Diabetic retinopathy, the leading cause of irreversible blindness in the working population in the Western world, encompasses both vascular and neural dysfunction (1). Diabetes mellitus leads to alterations in the perfusion and permeability of the retinal vasculature, resulting in retinal ischemia and/or edema, with loss of reading vision when these events occur in the central macular region (2). Diabetic retinopathy is also a degenerative disease of the neural retina, associated with alterations in neuronal function prior to the onset of clinical vascular disease (3). In advanced, proliferative diabetic retinopathy, an angiogenic, VEGF-mediated response with retinal neovascularization ensues, placing the eye at further risk for severe visual loss due to the development of vitreous hemorrhage or traction retinal detachment (4). Although many cases of diabetic retinopathy may be amenable to treatment with laser photocoagulation or vitrectomy surgery, such efforts may not prevent irreversible vascular or neuronal damage, thereby underscoring the need for early intervention.

The duration and severity of hyperglycemia is the single most important factor linked to the development of diabetic retinopathy. The degree of hyperglycemia is the major alterable risk factor for both the development and progression of diabetic retinopathy, both in type 1 and type 2 diabetes, as seen in the Diabetes Control and Complications Trial (DCCT) (5) and in the United Kingdom Prospective Diabetes Study (UKPDS) (6), respectively. Additional established risk factors for the acceleration of diabetic retinopathy include hypertension and hyperlipidemia, with several clinical studies demonstrating benefit in the treatment of diabetic retinopathy with intensive blood pressure control and lipid lowering therapy (7-13).

One metabolic consequence of chronic hyperglycemia is the accelerated formation of advanced glycation endproducts (AGEs), whose accumulation in diabetic tissues is enhanced not only by elevated glucose but also by oxidant stress and inflammatory stimuli (14). In the setting of diabetic retinopathy, AGEs, especially N^{ε}-(carboxymethyl)lysine (CML) adducts, have been detected within retinal vasculature and neurosensory tissue of diabetic eyes (15). Multiple consequences of AGE accumulation in the retina have been demonstrated, including upregulation of VEGF, upregulation of NFκB, and increased leukocyte adhesion in retinal microvascular endothelial cells (16-18). In diabetic patients, AGEs also accumulate within the vitreous cavity and may result in characteristic structural alterations sometimes referred to as “diabetic vitreopathy” (19, 20). Support for a role for AGEs as a contributing factor to the pathogenesis of diabetic retinopathy has been drawn from studies in animals with inhibitors of AGE formation (21, 22). In a 5-year study in diabetic dogs, administration of aminoguanidine prevented retinopathy; similar beneficial effects in the retinal vasculature of diabetic rats have been observed with other inhibitors of AGE formation, including pyridoxamine and benfotiamine (23, 24).

AGEs exert cell-mediated effects via RAGE, a multiligand signal transduction receptor of the immunoglobulin superfamily (25). Coinciding with pathologic changes in tissues, RAGE expression increases dramatically, with AGE ligands further upregulating receptor expression to magnify local cellular responses (26). RAGE also binds the proinflammatory mediators, the S100/calgranulins and amphoterin (27, 28), and is an endothelial cell adhesion receptor capable of promoting leukocyte recruitment through interaction with the integrin Mac-1 (29). Consequences of ligand-RAGE interaction include increased expression of VCAM-1, vascular hyperpermeability, enhanced thrombogenicity, induction of oxidant stress and abnormal expression of eNOS, all pathogenetic mechanisms that potentially contribute to the ischemic and vasopermeability events of diabetic retinopathy (30, 31).

### SUMMARY OF THE INVENTION

This invention provides method for treating diabetic retinopathy in a subject afflicted therewith, comprising administering to the subject's eyes a therapeutically effective amount of soluble RAGE or a derivative thereof, thereby treating diabetic retinopathy in the subject.

This invention further provides a method for inhibiting the onset of diabetic retinopathy in a subject afflicted therewith, comprising administering a prophylactically effective amount of soluble RAGE or a derivative thereof to the subject's eyes, thereby inhibiting the onset of diabetic retinopathy in the subject.

### BRIEF DESCRIPTION OF THE FIGURES

FIGS. 1A-E

Retinal elastase digest results among diabetic, hyperlipidemic, and littermate control mice at age 6 months. The development of acellular capillaries (A, B) is accelerated in the retinas of hyperglycemic, hyperlipidemic (HGHL) mice, with significantly more acellular capillaries present per unit area compared normoglycemic mice (normoglycemic, normolipidemic [NGNL]; normoglycemic, hyperlipidemic [NGHL]) and hyperglycemic, normolipidemic (HGNL) mice. Pericyte ghosts (C, D) were also increased in the retinas of hyperglycemic, hyperlipidemic (HGHL) mice compared to normoglycemic, normolipidemic (NGNL) littermates at age 6 months. Capillary outpouching (arrow, E), suggesting early microaneurysm formation, was observed in the retinal vasculature of HGHL mice. An intercapillary bridge, a normal feature of retinal digests not included in analysis, is also visible in this photograph (arrowhead). Results are expressed as mean ±SEM. Scale bat=50 μm. *P<0.05. **P<0.01.

FIGS. 2A-F

RAGE expression in the retina of normoglycemic, normolipidemic (NGNL) and hyperglycemic, hyperlipidemic (HGHL) mice. RAGE immunofluorescence (A, D, color not shown) colocalizes with vimentin (B, E, color not shown), a marker of Müller cells (arrows) in both NLNG and HGHL mice (C and F). The extension of Müller cells from the internal to the external limiting membranes of the neurosensory is highlighted with RAGE's expression (A, D). ILM, inner limiting membrane; IPL, inner plexiform layer; INL, inner nuclear layer; ONL, outer nuclear layer; ELM, external limiting membrane. Scale bar=50 μm.

FIGS. 3A-F

RAGE, GFAP (glial fibrillary acidic protein), and CD31 immunohistochemistry of the retina of hyperglycemic, hyperlipidemic mice. RAGE expression is prominent in Müller cell processes, particularly their internal footplates (A, D; color not shown, arrow heads) and is not observed in adjacent astrocytes (B, C; color not shown, arrows). The intimate vasoglial relationship of the RAGE-expressing Müller cell (color not shown, D) with the vascular endothelium of a retinal capillary (color not shown, E) is observed in Figure F. ILM, inner limiting membrane; IPL, inner plexiform layer; INL, inner nuclear layer. Scale bar=25 μm.

FIGS. 4A-H

RAGE (color not shown) and AGE (color not shown) immunohistochemistry of the vitreoretinal interface in normoglycemic, normolipidemic mice (A, B, C) and hyperglycemic, hyperlipidemic mice (E, F, G). AGEs are detected within the vitreous cavity, posterior vitreous cortex, and internal limiting membrane of the retina (color not shown, B, F). The internal footplates of RAGE-expressing Müller cells (color not shown, A, E) are immediately adjacent to AGEs in the internal limiting membrane (C, G). Controls (D and H). Vit, vitreous cavity; ILM, inner limiting membrane; GCL, ganglion cell layer; IPL, inner plexiform layer; INL, inner nuclear layer. Scale bar=25 μm.

FIGS. 5A-B

Retinal AGE ELISA and RAGE mRNA transcripts. Retinal AGEs accumulated in the retinas of hyperglycemic mice; both hyperglycemic, normolipidemic (HGNL) mice and hyperglycemic, hyperlipidemic (HGHL) mice had significantly increased AGEs compared to normoglycemic, normolipdemic (NGNL) littermates (A). RAGE mRNA expression in the retina was increased in the setting of hyperglycemia and AGE accumulation. RAGE transcripts were highest in the retinas of hyperglycemic, hyperlipidemic (HGHL) mice, with a nearly two fold elevation compared to basal levels in normoglycemic, normolipidemic (NGNL) littermates as well as a significant increase compared to normoglycemic, hyperlipidemic (NGHL) mice (B). Results are expressed as mean ±SEM. *P<0.05. **P<0.01.

FIGS. 6A-B

Effect of RAGE antagonism upon vascular changes in HGHL mice. Soluble RAGE-treated mice developed significantly less acellular capillaries (A) and pericyte ghosts (B) in the retina compared to untreated HGHL mice. Treatment of these mice also reduced the latency delays observed in the oscillatory potentials, with a significant reduction in the implicit times of OP2, OP3 and Σ OPs (the summation of OPs). *P<0.05. NGNL (normoglycemic, normolipidemic mice); HGHL (hyperglycemic, hyperlipidemic mice); sRAGE (soluble RAGE-treated HGHL mice).

FIG. 7

Amino acid sequence of bovine RAGE (Genbank Accession No. M91212).

FIG. 8

Nucleotide sequence of bovine RAGE (Genbank Accession No. M91212).

FIG. 9

Amino acid sequence of human RAGE (Genbank Accession No. M91211).

FIG. 10

Nucleotide sequence of human RAGE (Genbank Accession No. M91211).

FIG. 11

Amino acid sequence of mouse RAGE (Genbank Accession No. L33412).

FIG. 12

Nucleotide sequence of mouse RAGE (Genbank Accession No. L33412).

FIG. 13

Amino acid sequence for human soluble RAGE.

### DETAILED DESCRIPTION OF THE INVENTION

### Terms

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, intraocularly, topically and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“Agent” shall mean any chemical entity, including, without limitation, a glycomer, a protein, an antibody, a lectin, a nucleic acid, a small molecule, and any combination thereof.

“Degrade”, with respect to AGE, shall mean to cause the cleavage of one or more chemical bonds within the AGE, so as to render the AGE incapable, or less capable, of binding to RAGE.

A “derivative” of soluble RAGE (sRAGE) shall include, without limitation, a polypeptide or polypeptide-containing composition of matter, other than sRAGE itself, which comprises all or a portion of sRAGE. In one embodiment, the derivative is a polypeptide comprising a portion of sRAGE (e.g. an N-terminal portion, such as the V-domain). In another embodiment, the derivative is a fusion protein comprising an N-terminal portion of soluble RAGE, fused to an Fc domain-containing portion of an immunoglobulin (Ig). Examples of fusion proteins are described below.

“Modulate”, with respect to the binding between AGE and RAGE, shall include, without limitation, decreasing such binding by (i) inhibiting such binding from occurring (e.g. through competitive inhibition), (ii) causing disassociation of AGE already bound to RAGE, and/or (iii) causing degradation of AGE (which is already bound to RAGE or which would otherwise bind to RAGE).

“Prophylactically effective amount” means an amount sufficient to inhibit the onset of a disorder or a complication associated with a disorder in a subject.

“Subject” shall mean any organism including, without limitation, a mammal such as a mouse, a rat, a dog, a guinea pig, a ferret, a rabbit and a primate (human or non-human). In the preferred embodiment, the subject is a human being.

“Therapeutically effective amount” of an agent means an amount of the agent sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art. In one embodiment, the therapeutically effective amount is from about 1 mg of agent/subject to about 1 g of agent/subject per dosing. In another embodiment, the therapeutically effective amount is from about 10 mg of agent/subject to 500 mg of agent/subject. In a further embodiment, the therapeutically effective amount is from about 50 mg of agent/subject to 200 mg of agent/subject. In a further embodiment, the therapeutically effective amount is about 100 mg of agent/subject. In still a further embodiment, the therapeutically effective amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject.

“Treating” a disorder shall mean slowing, stopping or reversing the disorder's progression. In the preferred embodiment, treating a disorder means reversing the disorder's progression, ideally to the point of eliminating the disorder itself.

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Gutamic acid; G=Gly=Glycine; H=His=Histidine; I=Ile=Isoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=,Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

### EMBODIMENTS OF THE INVENTION

This invention provides a method for treating diabetic retinopathy in a subject afflicted therewith, comprising administering to the subject's eyes a therapeutically effective amount of soluble RAGE or a derivative thereof, thereby treating diabetic retinopathy in the subject.

In one embodiment, the subject is a rat, a dog, a mouse, a non-human primate or a human. In another embodiment, the soluble RAGE or derivative thereof (i.e. an sRAGE/Ig fusion protein) is admixed with a pharmaceutically acceptable carrier.

In one embodiment, the soluble RAGE or derivative thereof is administered via injection into the subject's eyes. In another embodiment, the soluble RAGE or derivative thereof is injected in or around the footplate region of the Müller cells in the subject's eyes. In a further embodiment, the soluble RAGE or derivative thereof is administered to the subject's eyes in the form of one or more pellets. Pellets for use in ocular drug administration are known (e.g. VITRASERT® for CMV treatment and RETISERT® for inflammation).

In one embodiment, the subject is receiving one or more forms of treatment for diabetic retinopathy in addition to soluble RAGE or a derivative thereof. For example, a therapeutically effective amount of an agent, other than soluble RAGE or a derivative thereof, that modulates the binding between AGE and RAGE in the subject's eyes, is further administered to the subject's eyes. In one embodiment, the agent inhibits the binding between AGE and RAGE in the subject's eyes. In another embodiment, the agent disassociates bound AGE from RAGE in the subject's eyes. In another embodiment, the agent degrades one or more AGES in the subject's eyes.

In one embodiment, the agent is an enzyme, such as dispase. In another embodiment, the agent is N-phenyl-thiazolium, or a bromide or chloride salt thereof.

In one embodiment of this method, the agent is administered topically to the subject's eyes. In another embodiment, the agent is administered via injection into the subject's eyes. In another embodiment, the agent is injected in or around the footplate region of the Müller cells of the subject's eyes. In a further embodiment, the agent is administered to the subject's eyes in the form of one or more pellets. The agent and sRAGE or derivative thereof can be administered together (i.e. in the same composition), concurrently or separately.

This invention further provides a method for inhibiting the onset of diabetic retinopathy in a subject afflicted therewith, comprising administering a prophylactically effective amount of soluble RAGE or a derivative thereof to the subject's eyes, thereby inhibiting the onset of diabetic retinopathy in the subject.

In one embodiment, the subject is a rat, a dog, a mouse, a non-human primate or a human. In another embodiment, the soluble RAGE or derivative thereof (i.e. an sRAGE/Ig fusion protein) is admixed with a pharmaceutically acceptable carrier.

In one embodiment, the soluble RAGE or derivative thereof is administered via injection into the subject's eyes. In another embodiment, the soluble RAGE or derivative thereof is injected in or around the footplate region of the Müller cells in the subject's eyes. In a further embodiment, the soluble RAGE or derivative thereof is administered to the subject's eyes in the form of one or more pellets. Pellets for use in ocular drug administration are known (e.g. VITRASERT® for CMV treatment and RETISERT® for inflammation).

In one embodiment, the subject is receiving one or more forms of treatment for diabetic retinopathy in addition to soluble RAGE or a derivative thereof. For example, a therapeutically effective amount of an agent, other than soluble RAGE or a derivative thereof, that modulates the binding between AGE and RAGE in the subject's eyes, is further administered to the subject's eyes.

In one embodiment, the agent inhibits the binding between AGE and RAGE in the subject's eyes. In another embodiment, the agent disassociates bound AGE from RAGE in the subject's eyes. In another embodiment, the agent degrades one or more AGES in the subject's eyes.

In one embodiment, the agent is an enzyme, such as dispase. In another embodiment, the agent is N-phenyl-thiazolium, or a bromide or chloride salt thereof.

In one embodiment of this method, the agent is administered topically to the subject's eyes. In another embodiment, the agent is administered via injection into the subject's eyes. In another embodiment, the agent is injected in or around the footplate region of the Müller cells of the subject's eyes. In a further embodiment, the agent is administered to the subject's eyes in the form of one or more pellets. The agent and sRAGE or derivative thereof can be administered together (i.e. in the same composition), concurrently or separately.

Nucleotide and Amino Acid Sequences of RAGE

The nucleotide and protein (amino acid) sequences for RAGE (both human and murine and bovine) are known. The following references which recite these sequences are incorporated by reference: Schmidt et al, J. Biol. Chem., 267:14987-97, 1992; and Neeper et al, J. Biol. Chem., 267:14998-15004, 1992.

Soluble RAGE

The following are examples of forms of soluble RAGE: mature human soluble RAGE, mature bovine soluble RAGE, and mature murine soluble RAGE. Representative portions of sRAGE include, but are not limited to, peptides having an amino acid sequence which corresponds to amino acid numbers (2-30), (5-35), (10-40), (15-45), (20-50), (25-55), (30-60), (30-65), (10-60), (8-100), 14-75), (24-80), (33-75), (45-110) of human sRAGE protein. The 22 amino acid leader sequence of immature human RAGE is Met Ala Ala Gly Thr Ala Val Gly Ala Trp Val Leu Val Leu Ser Leu Trp Gly Ala Val Val Gly.

sRAGE/Ig Fusion Proteins

Examples of fusion proteins include polypeptides comprising (i) the V-domain of sRAGE linked to the CH2 and CH3 domains (i.e. Fc domain) of an Ig, and (ii) the V-domain and C1 domain of sRAGE linked to the CH2 and CH3 domains of an Ig. In these two examples, the fusion of part (i) can comprise, for example, about 250 amino acid residues (with about 136 residues belonging to the sRAGE V-domain), and the fusion protein of part (ii) can comprise, for example, about 380 amino acid residues. In one embodiment of each of the fusion proteins of parts (i) and (ii), the sRAGE V-domain-containing portion of the fusion protein comprises an amino acid sequence (e.g. about 30 amino acid residues) which permits binding to Aβ peptide. Such sequence can be, for example, A-Q-N-I-T-A-R-I-G-E-P-C-V-L-K-C-K-G-A-P-K-K-P-P-Q-R-L-E-W-K (see, e.g. U.S. Pat. No. 6,555,651 (58)), or the first ten residues thereof.

This invention will be better understood from the Experimental Details which follow. However, one skilled in the art will readily appreciate that the specific methods and results discussed are merely illustrative of the invention as described more fully in the claims which follow thereafter.

### Experimental Details

### Synopsis

The Receptor for AGEs (advanced glycation endproducts) has been implicated in the pathogenesis of diabetic complications. This study characterizes the role of the RAGE axis in a murine model of nonproliferative diabetic retinopathy (NPDR).

Hyperlipidemic apoE −/− mice were first bred into the hyperglycemic db/db background, and hyperlipidemia accelerating structural vascular changes in diabetic retinas that exhibit neuronal dysfunction was observed. The RAGE axis was localized and quantified, specifically AGE ligands and their cellular receptor RAGE, in the eyes of these mice. The findings provide new insights into the role of the RAGE axis in the pathogenesis of early diabetic retinopathy.

### Materials and Methods

Generation of Mouse Colony

To generate the apoE −/− db/db mice, apoE −/− mice were first backcrossed six generations into mice heterozygous for the diabetes spontaneous mutation (Lepr db). As the homozygous db/db mouse is sterile, apoE −/− m/db offspring were ultimately bred to generate apoE −/− db/db mice. Initially, male mice heterozygous for the diabetes spontaneous mutation (Lepr db) in the leptin receptor gene on Chromosome 4 (BKS.Cg-m+/+Lepr db, former name C57BLK/J-m+/+Lepr db, Type JAX® GEMM TM Strain—Spontaneous Mutation Congenic, Stock number 000642; Jackson Laboratory, Bar Harbor, Me.) were crossed with female mice homozygous for the ApoE tm1Unc mutation in chromosome 7 (B6.129P2-Apoe tm1Unc, former name C57BL/6J− Apoe tm1Unc, Type JAX® GEMM TM Strain-Targeted Mutation Congenic, Stock number 002052; Jackson Laboratory) at about 8 weeks of age. All mice were fed normal rodent chow (5053, PMI Nutrition International, Inc., St. Louis, Mo.) and exposed to a 12 hour light-dark cycle. All offspring were heterozygous for the apoE mutation. The genotype of their offspring was identified by PCR using primers from Invitrogen Corp. (Carlsbad, Calif.). The heterozygous mice from different parents were again crossed at 8 weeks of age. Mice homozygous for the ApoE tm1Unc mutation and heterozygous for the Lepr db mutation (apoE −/− db/m) were used as breeders and were crossed with one another to breed the double knock-out apoE −/− db/db mice. Control mice were littermates obtained from the same colony: apoE +/+, m/db mice (homozygous for the wild type allele ApoE tm1Unc and heterozygous for the db mutation) which are normoglycemic, nonobese littermates; apoE +/+db/db mice (homozygous for the wild type allele ApoE tm1Unc and homozygous for the Lepr db mutation) which are hyperglycemic, normolipidemic littermates. Glucose measurements were performed during the course of generation of the colony using a glucometer (Freestyle®, Therasense, Alameda, Calif.). Cholesterol measurements were performed using the Infin Cholesterol Liquid Stable Reagent kit (Thermo Electron Corp, Waltham, Mass.). The generation of the colony and all experiments were done in agreement with ARVO statement for the use of animals in ophthalmic and vision research and were approved by the Institutional Animal Care and Use Committee at Columbia University.

Elastase Retinal Digest

Elastase digest with histopathological vascular analysis was performed upon 35 mice at age 6 months, including analysis of the following phenotypes: apoE +/+db/m (n=7; normoglycemic, normolipidemic [NGNL]); apoE −/− db/m (n=8; normoglycemic, hyperlipidemic [NGHL]); apoE +/+db/db (n=7; hyperglycemic, normolipidemic [HGNL]); apoE −/− db/db (n=13; hyperglycemic, hyperlipidemic [HGHL]). At the time of sacrifice, the eyes were enucleated and placed in 10% formalin for 2 days. After fixation, the retina was gently dissected away from the neurosensory retina under microscopic observation. The neurosensory retina was placed in distilled water overnight to remove fixative. The elastase digestion method described by Layer was then performed (32). After mounting of the vascular specimen on a slide, periodic acid schiff and hematoxylin staining of the vascular network and nuclei was performed. The specimens were then analyzed using an Axioskop 2 Plus microscope with digital capture (Carl Zeiss. MicroImaging Inc., Thornwood, N.Y.) for the presence of acellular capillaries and pericyte ghosts. Acellular capillaries were at least one-third thickness of normal capillary width, and intercapillary bridges were excluded from analysis (33). The examiner was masked to the nature of the specimen during the assessment of pathology. As vascular lesions may be distributed non-uniformly, the entire retina was scanned during this process, and images were pasted into a single image within Adobe Photoshop version 7.0 (Adobe Systems Inc., San Jose, Calif.) to obtain an image of whole mounted retina for area calculations. The virtual area of each prepared retina was measured with OphthaVision Imaging System version 3.25 (MRP Group Inc., Lawrence, Mass.). The number of acellular capillaries and pericyte ghosts for each digest was divided by the area scanned. The data obtained were analyzed with frequency and descriptive statistics as described below.

Electrophysiology

Electroretinograms (ERGs) were performed upon the following age-matched, 6 month old, littermates: normoglycemic, normolipidemic wild type mice (NGNL; apoE +/+db/m; n=18); normoglycemic, hyperlipidemic mice (NGHL; apo E −/− db/m; n=11); hyperglycemic, normolipidemic mice (HGNL; apoE +/+db/db; n=8); and hyperglycemic, hyperlipidemic mice (HGHL; apoE −/− db/db; n=14). The mice were dark-adapted overnight before each experiment, and the ensuing procedures were performed under dim red light in a darkroom. The mice were anesthetized with a mixture of 50 mg/kg ketamine and 5 mg/kg xylazine administered intraperitoneally. The right eye pupil was dilated with drops of 2.5% phenylephrine-hydrochloride and 0.5% tropicamide. The electroretinogram (ERG) responses were amplified and averaged by a computerized data acquisition system (PowerLab; ADInstruments, Colorado Springs, Colo.). Once anesthetized, the mouse was placed on a heating block, and body temperature was maintained near 37° C. The mouse was placed in a centered position at the edge of a Ganzfeld dome. A rectal thermometer was placed in the mouse and checked throughout the recording. A ground electrode was inserted in the right leg and the reference electrode was inserted in the forehead. The data collected and analyzed included all the above and temperature of the animal during the experiment, a- and b-wave latency and amplitude, oscillatory potentials 1 (OP1), 2 (OP2) and 3 (OP3) implicit time and amplitude as previously described (34, 35). The data obtained were analyzed with frequency and descriptive statistics as described below.

Immunochemical Staining

Eyes from 6-mo-old mice were fixed overnight in 4% phosphate-buffered paraformaldehyde and embedded in paraffin. The 4 mm paraffin sections were deparaffinized and heated in citrate buffer using a microwave for 15 minutes. After pretreatment with PBS containing 5% normal goat serum (Jackson ImmunoResearch Laboratories Inc., West Grove, Pa.), 0.5% BSA and 0.1% Triton X-100 for 30 minutes at room temperature (RT), sections were incubated with anti-mouse RAGE antibody (36) (1:100), anti-AGE antibody (36) (1:100), anti-vimentin antibody (1:200, Santa Cruz Biotechnology Inc., Santa Cruz, Calif.), antiglial fibrillary acidic protein (GFAP) antibody (1:100, Chemicon International, Inc., Temecula, Calif.), or anti-CD31 antibody (1:200, Pharmingen, San Diego, Calif.) for 1 hour at RT and then overnight at 4° C. After rinsing with PBS, sections were incubated for 1 hour at RT with secondary antibody conjugated to Alexa Fluor® 488 (Molecular Probes Inc., Eugene, Oreg.) or Alexa Fluore 546 (Molecular Probes Inc.) All antibodies were diluted in PBS containing 0.5% goat serum, 0.5% BSA and 0.1% Triton X-100. Rabbit or chicken serum was used instead of primary antibody for negative controls. The retina was examined with a Nikon Eclipse E800 microscope (Nikon Instruments Inc., Meville, N.Y.) equipped with confocal laser scanning system (Radiance2000; Bio-Rad Laboratories, Hercules, Calif.). Images were captured and processed using BioRad LaserSharp 2000 software (Bio-Rad Laboratories).

Autofluorescence and ELISA of Retinal AGEs

Five mice from each group were sacrificed. Whole retina was homogenized in 0.1 ml of PBS with 0.1% Triton X-100 at 0° C. Samples were centrifuged at 20,000×g for 5 minutes at 4° C. Protein concentration was determined using BSA as a standard. The protein level in supernatant was adjusted to 1.6 mg/ml and used for cellular protein autofluorescence assay. The pellet, mostly extracellular matrix (ECM), was washed with 20 mM phosphate buffer, pH 7.0, with 10 mM EDTA, and digested with 20 μl of 25 Units/ml papain (Sigma P5306, Sigma, St. Louis, Mo.) in 20 mM phosphate buffer, pH 7.0, mM EDTA, 20 mM cysteine at 37° C. After 24 hours, another 20 μl of papain solution was added, and the incubation was continued for 24 hours. The supernatant was utilized for the measurement of ECM autofluorescence and ELISA of AGEs after appropriate dilution. Fluorescence intensities were measured on an Applied Biosystems Multi-Well Plate Reader—CytoFluor 4000 (Foster City, Calif.) using 360±40/460±40 nm excitation/emission wavelengths. These excitation/emission wavelengths allow for detection of well-defined AGEs (37, 38). Fluorescence values were expressed in fluorescence intensity per 0.1 mg cellular protein or its equivalent retina size for ECM. For immunochemical measurement of AGEs in ECM, a noncompetitive ELISA was employed. The wells (96-well Nunc-Immuno™ Plate, Nalge Nunc International, Rochester, N.Y.) were coated with BSA control, AGE-BSA standard (36) and biological samples in 0.1 ml of 50 mmol/L carbonate buffer (pH 9.6) at 4° C. overnight. The wells were then washed with PBS containing 0.05% Tween 20 (washing buffer) and blocked at room temperature with 0.3 ml of 1% BSA and 5% rabbit serum in PBS (blocking buffer) for 1 hour. After washing, the wells were incubated with anti-AGE antibody (36) in blocking buffer for 3 hours at room temperature followed by washing and secondary antibody (rabbit anti-chicken IgY-HRP, Biomeda Corp, Foster City, Calif.) for 1 hour at room temperature. The wells were then washed again and developed with 0.1 ml of peroxidase substrates (o-phenylenediamine tablets, Sigma) in dark at room temperature. The absorbance at 490 nm was measured after adding 0.05 ml of stopping solution (2M H₂SO₄) at 10 minutes.

Quantitative Real-Time PCR

At least five mice of each group were sacrificed. Retinas were isolated and stored in pairs at −80° C. in RNAlater™ (Ambion, Inc., Austin, Tex.). Total RNA was prepared using RNeasy Minikijt (QIAGEN Inc., Valencia, Calif.). After quantification at OD₂₆₀ total RNA was analyzed using RNA Nano LabChips on a 2100 Bioanalyzler (Agilent Technologies, Palo Alto, Calif.) to assess RNA quality. Only samples showing minimal degradation were used cDNA was synthesized using TaqMan Reverse Transcription Reagents Kit (Applied Biosystems, Foster City, Calif.) according to manufacturer's instructions. Primers and probes for β-actin and RAGE were designed using Primer Express® software (Applied Biosystems). To confirm specific amplification of the target mRNA, an aliquot of the PCR product was analyzed using gel electrophoresis. The sequences of the primers and probe were as follows: for β-actin, 5′-ACG GCC AGG TCA TCA CTA TTG-31 (forward), 5′-TGG ATG CCA CAG GAT TCC AT-3′ (reverse) and 5′-6FAM-ACG TCT ACC AGC GAA GCT ACT GCC GTC-TAMRA-3′ (probe); for RAGE, 5′-GGA CCC TTA GCT GGC ACT TAG A-3′ (forward), 5′-GAG TCC CGT CTC AGG GTG TCT-3′ (reverse) and 5′-6FAM-ATT CCC GAT GGC AAA GAA ACA CTC GTG-TAMRA-3′ (probe) (Applied Biosystems). Real-time PCR was conducted using ABI PRISM 7900HT Sequence Detection System and results were analyzed using the 2^{-ΔΔc}T method (39). Experiments were repeated 3 times, and statistical analysis was performed as described below.

Administration of Soluble RAGE

Soluble RAGE, the extracellular two-thirds of the receptor, binds AGEs and interferes with their ability to bind and activate cellular RAGE. Preparation, characterization, and purification of sRAGE were performed using a baculovirus expression system using Sf9 cells (Clontech, Palo Alto, Calif.; Invitrogen Corp.) as previously described (36). Purified murine sRAGE (a single-band of about 40 kDa, by Coomassie-stained SDS-PAGE) was dialyzed against PBS; made free of detectable endotoxin, based on the Limulus amebocyte assay (E-Toxate; Sigma) after passage onto Detoxi-Gel columns (Pierce Chemical Co., Rockford, Ill.); and sterile-filtered (0.2 μm). Daily doses of 100 μg of sRAGE were administered based upon previous-dose response studies (27).

Statistical Analysis

To analyze the vascular, neuronal, and experimental data among the four groups, two-factor Analysis of Variance (ANOVA) model was used. The two factors considered were glucose (normal/high) and lipid (normal/high). Interactions were tested for all analyses but none were found. A one-way Analysis of Variance (ANOVA) was also used to compare the four groups in analyzing the AGE ELISA and autofluorescence data and the RAGE q-PCR data. For the experiment involving treatment with sRAGE, a one-way ANOVA was used to compare the three (3) groups, NGNL, HGHL, and sRAGE. If a difference was found among the groups (p<0.05), a posthoc analysis using the Duncan test was performed. All data was analyzed using SAS system software (SAS Institute Inc., Cary, N.C.).

### Results

Hyperlipidemia Accelerates the Development of Vascular Lesions of Early Diabetic Retinopathy in Hyperglycemic Mice

The serum levels of glucose and cholesterol for each of the four groups is presented in Table 1.

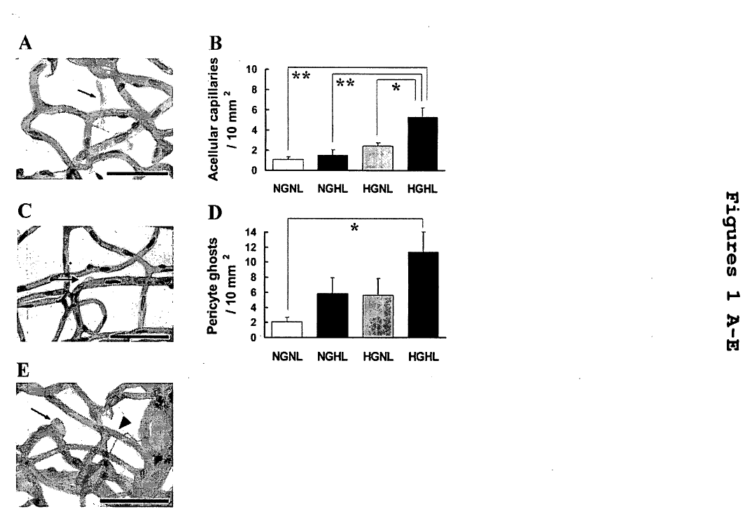

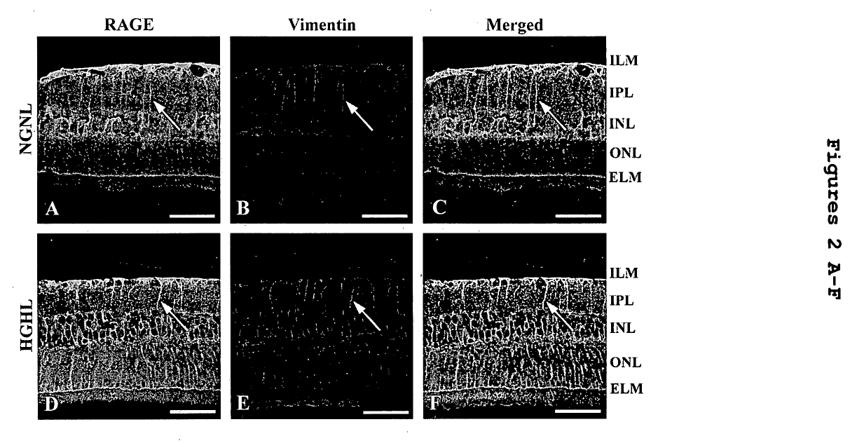

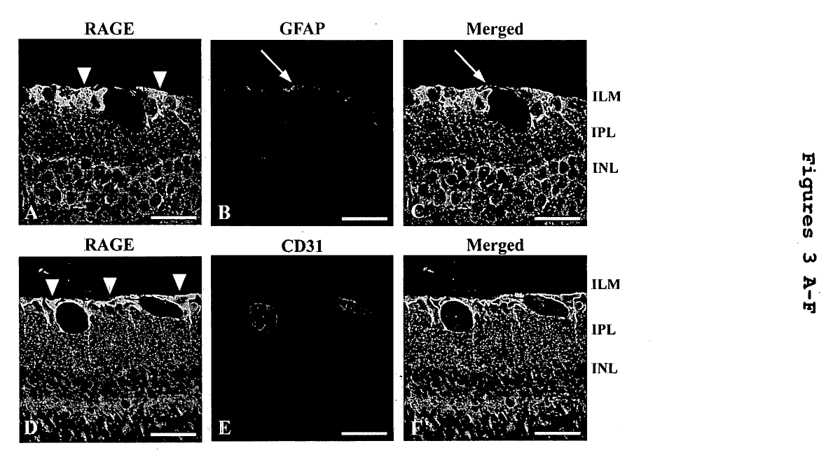

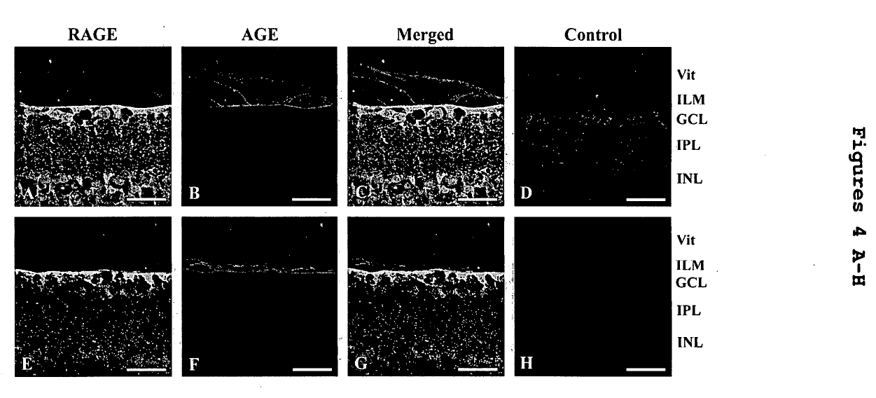

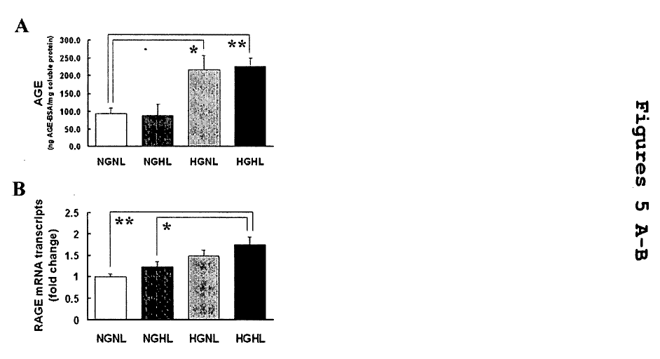

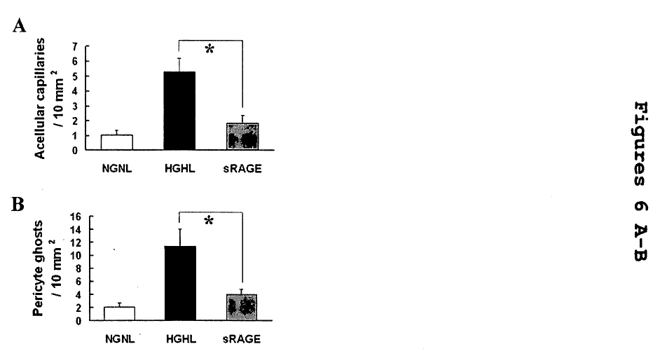

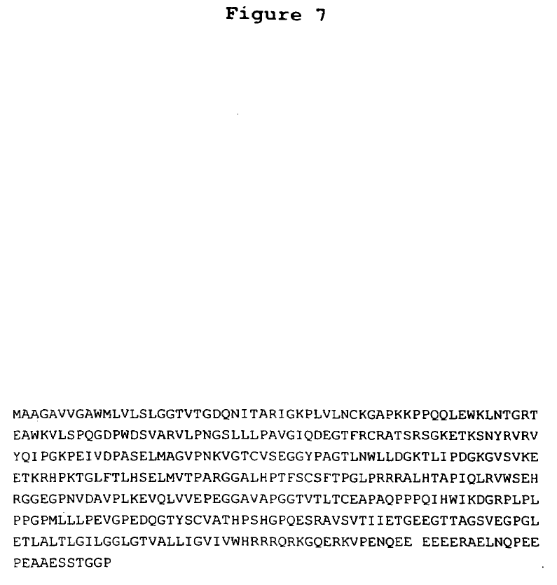

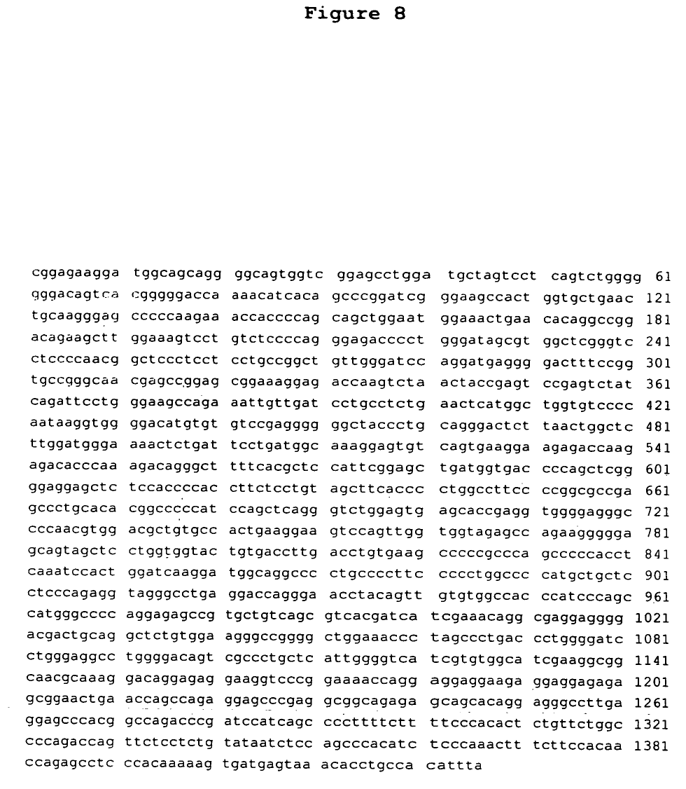

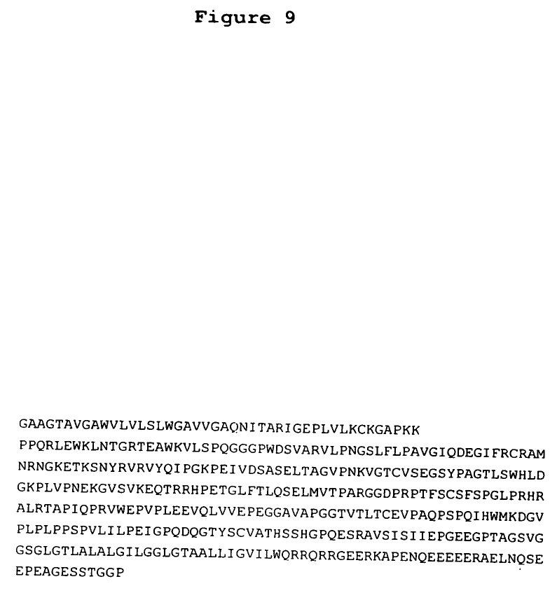

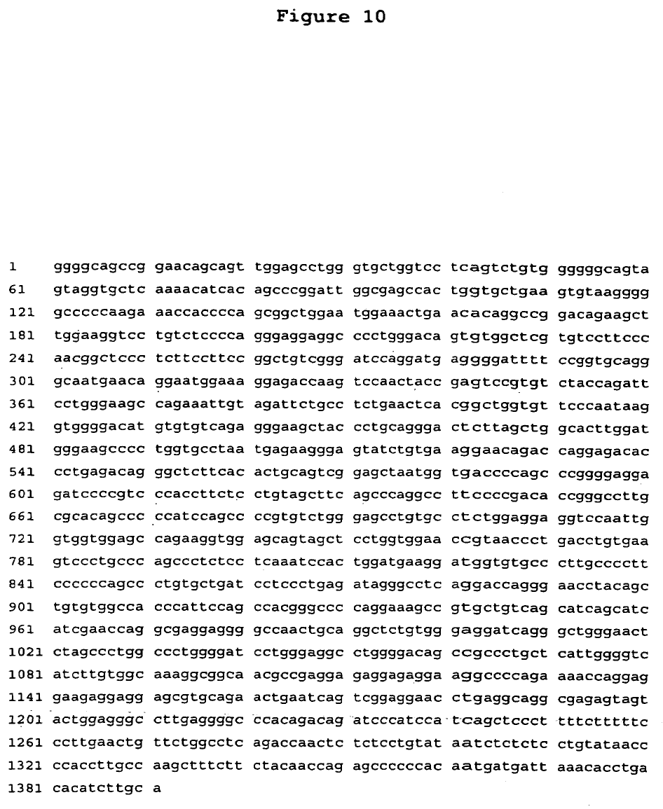

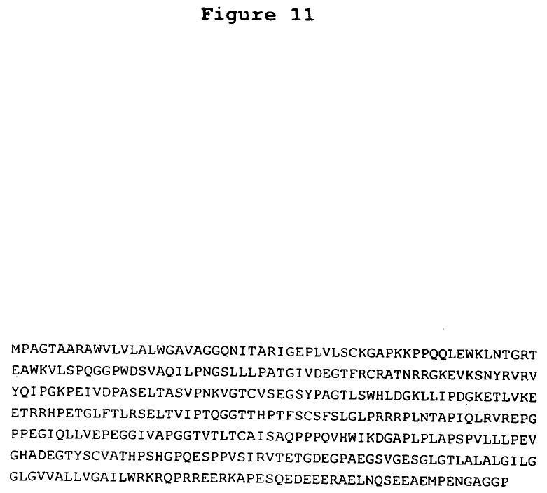

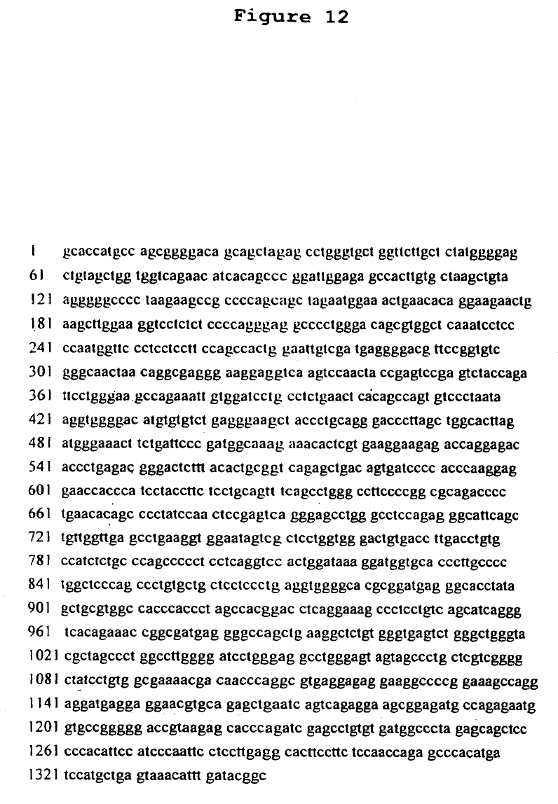

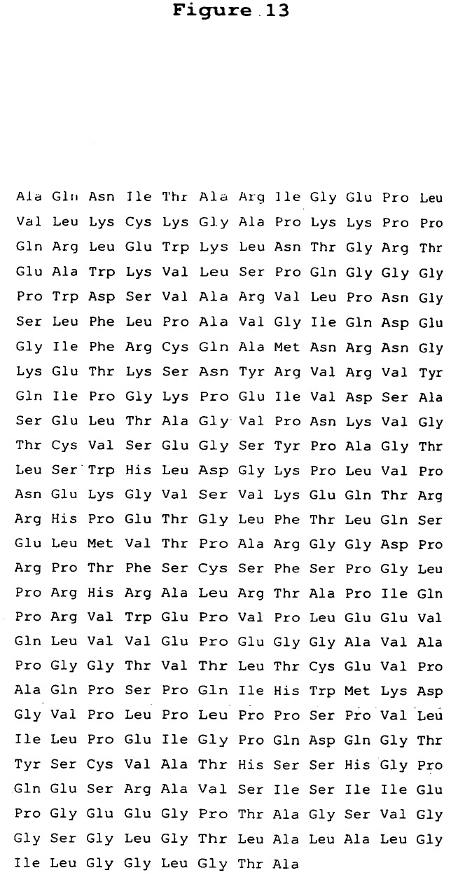

The impact of introduction of hyperlipidemia into the hyperglycemic db/db background was first examined on vascular properties in the retina. At age 6 months, the retinas of hyperglycemic, hyperlipidemic (HGHL, apoE −/− db/db) mice displayed the most significant capillary lesions of NPDR (FIG. 1). While the eyes of HGNL mice exhibited some development of acellular capillaries within the retina, only the eyes of HGHL mice had a significantly higher number of acellular capillaries compared to all other groups (FIG. 1B). The development of pericyte ghosts was detectable in both hyperglycemic (HGNL) and hyperlipidemic (NGHL) phenotypes, but only in the HGHL mice was there a significant difference compared to NGNL controls (FIG. 1D). Only in HGHL mice was there evidence of capillary outpouching consistent with early microaneurysm formation (FIG. 1E).

### Hyperglycemic Mice Demonstrate Electrophysiologic Neural Dysfunction of the Inner Retina

Electrophysiologic testing at age 6 months revealed that hyperglycemia resulted in early inner retinal dysfunction of the retina detected by prolongation in the latencies of the b-wave and the oscillatory potentials (Table 2).

Specifically, there were significant hyperglycemia-induced delays in the implicit time of the b-wave and the oscillatory potentials OP1, OP2, and OP3 (Table 4). The ERG amplitudes were not significantly affected in this study, with hyperglycemic mice demonstrating a statistically significant decline only in the amplitude of the oscillatory potential Op1 (Tables 3 and 4). Hyperlipidemia alone did not induce statistically significant differences in any of the parameters recorded and studied (Table 4).

The RAGE Axis is Accentuated at the Vitreoretinal Interface

RAGE expression was predominantly localized to glial cells of the inner retina. Most of the RAGE-expressing cells within neural retina were consistent with the distribution of Müller cells and particularly their internal footplates. In merged images, RAGE-positive cells of the inner retina colocalized with vimentin expression, confirming Müller cell expression (FIG. 2). Glial fibrillary acidic protein (GFAP) expression in astrocytes of the inner retina revealed no evidence of colocalization with adjacent RAGE expression of Müller cell processes and footplates (FIG. 3A-C). Expression of RAGE was also detected adjacent to the microvasculature, suggesting intimate neurovascular localization for RAGE in the circulation of inner retina (FIG. 3D-E). AGEs were prominently detected within the vitreous cavity of the eye and particularly along the vitreoretinal interface including the internal limiting membrane (FIGS. 4B, F). AGEs were consistently detected within lens capsule and Bruch's membrane and occasionally within the basement membrane of the microvasculature (not shown). In AGE and RAGE merged images, AGE was localized to vitreous fibrils and the internal limiting membrane, where there was close apposition to the footplates of RAGE-expressing Müller cells (FIG. 4).

RAGE and its AGE Ligands are Increased in NPDR

The RAGE axis in this murine model of NPDR was quantified. As AGEs can accumulate within cellular protein as well as within the proteins of extracellular matrix (ECM), the autofluorescence of AGEs were assayed independently. As seen in Table 5, there was not a significant difference among groups with regard to AGE autofluorescence in cellular protein. In contrast, AGE autofluorescence increased in ECM in the setting of hyperglycemia, but only the retinas of HGHL mice had a significant fluorescent difference in compared to NGNL mice. To further quantify AGEs in the ECM, a noncompetitive ELISA was performed. It was revealed that AGE formation in the retinal ECM of hyperglycemic mice was significantly increased, both HGNL and HGHL (FIG. 5A). As RAGE expression may be amplified in the setting of its ligands (40), RAGE mRNA expression from whole retina was then examined by quantitative real-time PCR for each group. RAGE mRNA expression was increased in the retinas of hyperglycemic mice (glucose effect for two factor ANOVA: P<0.01); a significant increase was observed in HGHL mice compared to each group of normoglycemic mice (FIG. 5B). These studies demonstrate that RAGE axis comprising the cellular receptor and its AGE ligands is amplified in the diabetic retina, particularly in eyes with significant capillary lesions of NPDR (HGHL mice).

Antagonism of RAGE Reduces Vascular Lesions of Diabetic Retinopathy and Ameliorates Neuronal Dysfunction at 6 Months of Age

Based upon the upregulation of AGEs and RAGE in the HGHL group, the potential contribution of RAGE in the pathogenesis of vascular and neuronal perturbation was tested. Murine sRAGE was administered to 10 HGHL mice from age 8 weeks to age 6 months. The number of acellular capillaries per 10 mm² in the retinal digest of treated mice was significantly less than those observed in nontreated mice (FIG. 6A). In addition, there were significantly fewer pericyte ghosts in the retinas of treated mice compared to nontreated mice (FIG. 6B). Electrophysiologic studies demonstrated that prophylactic treatment with sRAGE reduced retinal neuronal dysfunction, with a statistically significant (p<0.05) reduction in the hyperglycemia-induced latency delays observed in OP2, OP3, and ΣOPs at 6 months of age (Table 6). Treatment with sRAGE had no significant effect upon the amplitudes of the b-wave and oscillatory potentials (data not shown).

TABLE 1
Glucose and cholesterol level at sacrifice (age 6 months)

| | NGNL | NGHL | HGNL | HGHL |
|---|---|---|---|---|
| Glucose | 121.3 ± 28.5 (15) | 113.8 ± 24.2 (18) | 452.6 ± 109.4 (13) | 455.5 ± 68.4 (10) |
| (mg/dl) | | | | |
| Cholesterol | 62.5 ± 14.4 (5) | 471.2 ± 72.4 (15) | 201.3 ± 30.4 (5) | 955.6 ± 149.1 (15) |
| (mg/dl) | | | | |

Data are expressed as the mean ± SD (n)

TABLE 2
ERG latencies of mice at age 6 months

| | Latency (ms) | | | |
|---|---|---|---|---|
| | | | | HGHL |
| | NGNL (n = 18) | NGHL (n = 10) | HGNL (n = 8) | (n = 14) |
| b-wave | 32.0 ± 2.0 | 32.4 ± 4.0 | 35.3 ± 3.5 | 34.5 ± 2.9 |
| OP1 | 23.4 ± 1.4 | 23.0 ± 2.1 | 25.4 ± 1.9 | 24.6 ± 1.7 |
| OP2 | 32.0 ± 2.0 | 31.7 ± 3.2 | 34.8 ± 2.5 | 33.8 ± 2.2 |
| OP3 | 42.6 ± 2.9 | 42.9 ± 5.4 | 45.4 ± 3.2 | 44.9 ± 3.1 |
| Σ OPs | 98.0 ± 6.2 | 97.5 ± 10.6 | 105.6 ± 7.3 | 103.3 ± 6.8 |

Data are expressed as the mean ± SD

TABLE 3
ERG amplitudes of mice at age 6 months

| | Amplitude (mV) | | | |
|---|---|---|---|---|
| | NGNL (n = 18) | NGHL (n = 10) | HGNL (n = 8) | HGHL (n = 14) |
| b- | 568.4 ± 163.2 | 517.0 ± 141.0 | 462.5 ± 138.9 | 489.3 ± 186.5 |
| wave | | | | |
| OP1 | 234.6 ± 62.9 | 210.5 ± 84.4 | 173.5 ± 52.6 | 175.6 ± 67.2 |
| OP2 | 244.5 ± 89.8 | 206.8 ± 96.6 | 219.4 ± 54.7 | 202.9 ± 68.2 |
| OP3 | 96.2 ± 50.6 | 80.0 ± 39.8 | 109.9 ± 32.6 | 96.0 ± 50.0 |
| Σ | 575.3 ± 191.8 | 497.3 ± 212.1 | 502.8 ± 109.7 | 474.5 ± 168.2 |
| OPs | | | | |

Data are expressed as the mean ± SD

TABLE 4
Two factor ANOVA analysis of ERG data from Tables 2 and 3

| | | p value | | |
|---|---|---|---|---|
| | | Glucose effect | Lipid effect | Interaction |
| b-wave | Latency | 0.004 | 0.805 | 0.516 |
| | Amplitude | 0.174 | 0.799 | 0.422 |
| OP1 | Latency | 0.001 | 0.216 | 0.649 |
| | Amplitude | 0.021 | 0.588 | 0.516 |
| OP2 | Latency | 0.001 | 0.376 | 0.675 |
| | Amplitude | 0.550 | 0.225 | 0.661 |
| OP3 | Latency | 0.031 | 0.933 | 0.744 |
| | Amplitude | 0.283 | 0.271 | 0.934 |
| Σ OPs | Latency | 0.004 | 0.545 | 0.685 |
| | Amplitude | 0.376 | 0.324 | 0.643 |

TABLE 5
Retinal AGE fluorescent intensities

| | Autofluorescence | | | |
|---|---|---|---|---|
| | NGNL | NGHL | HGNL | HGHL |
| Cellular protein | 1357 ± 149 | 1666 ± 182 | 1122 ± 194 | 1181 ± 161 |
| ECM | 2801 ± 673 | 2342 ± 531 | 3713 ± 1229 | 5259 ± 715* |

Data are expressed as the mean ± SE
*Significant (p < 0.05) compared to NGNL group

TABLE 6
sRAGE effect upon ERG latencies at age 6 months

| | Latency (ms) | | |
|---|---|---|---|
| | NGNL (n = 18) | HGHL (n = 14) | sRAGE (n = 10) |
| b-wave | 32.0 ± 2.0 | 34.5 ± 2.9 | 33.3 ± 2.5 |
| OP1 | 23.4 ± 1.4 | 24.6 ± 1.7 | 23.6 ± 1.4 |
| OP2 | 32.0 ± 2.0 | 33.8 ± 2.2 | 32.2 ± 2.0* |
| OP3 | 42.6 ± 2.9 | 44.9 ± 3.1 | 42.0 ± 3.0* |
| Σ OPs | 98.0 ± 6.2 | 103.3 ± 6.8 | 97.7 ± 6.1* |

Data are expressed as the mean ± SD
*Significant (p < 0.05) compared to HGHL group

### Discussion

The pathogenesis of diabetic retinopathy remains complex, but prolonged hyperglycemia is required to develop anatomic retinal vascular lesions in human diabetic retinopathy and most animal models of diabetic retinopathy (41). In this context, the db/db mouse, a well-characterized murine model of hereditary, insulin-resistant diabetes first detected in the progeny of the C57BLKS/J strain at the Jackson Laboratory and later characterized as being deficient in leptin receptor signaling was investigated (42). While the db/db mouse develops neuropathy and nephropathy, the anatomic retinal vascular findings, apart from basement membrane thickening, are less dramatic. Previous anatomic studies revealed acellular capillaries and pericyte ghosts at age 8 months in db/db mice, but these anatomic findings were variable and inconsistently present (Barile G R, et al. Iovs 2000; 41:ARVO Abstract 2156). Hyperlipidemia is associated with the severity of diabetic retinopathy (8-10), and successful treatment of hyperlipidemia in diabetic patients may retard the progression of retinopathy or improve it (11-13). For these reasons, the influence of hyperlipidemia upon the retinal findings of the db/db mouse model of diabetes mellitus was investigated, ultimately crossing it with mice carrying a mutation in the apoE gene that leaves them devoid of functioning apoE protein. It was observed that the classic anatomic retinal lesions of nonproliferative diabetic retinopathy developed at the highest rate in hyperglycemic, hyperlipidemic mice compared to the other groups, consistent with the burgeoning notion that hyperlipidemia accelerates the retinal vascular disease of diabetes mellitus. These results further support increasing evidence that dyslipidemia in diabetes mellitus independently contributes to the pathogenesis and severity of diabetic retinopathy, possibly via amplification of inflammatory mechanisms (43, 44).

While diabetic retinopathy is classically a microvascular disease of the retinal capillaries, diabetes may impair retinal neuronal function before the onset of visible vascular lesions. Numerous psychophysical and electrophysiological studies demonstrate early retinal neuronal dysfunction in diabetes mellitus, prior to the onset of the classic microvascular lesions of diabetic retinopathy (45, 46). In particular, Bresnick and colleagues have emphasized that alterations in the oscillatory potentials of the electroretinogram better predict the development of high-risk proliferative retinopathy than do clinical fundus photographs (47, 48). Pathological quantification of neural loss by Barber and colleagues showed apoptosis of retinal neurons and retinal atrophy, with loss of inner retinal thickness and cell bodies, in both diabetic rats and human subjects (49). Several investigators have noted other retinal neuronal alterations in early diabetes, including glial fibrillary acidic protein (GFAP) activation and glutamate transporter dysfunction in Müller cells (50, 51). In this study, it was demonstrated that chronic hyperglycemia caused significant implicit time delays of oscillatory potentials at 6 months that are comparable to previous studies in diabetes (52), while hyperlipidemia did not influence these electrophysiologic parameters. In conjunction with the histopathologic vascular changes observed above, this study supports the concept of early diabetic retinopathy as a neurovascular disease of the retina, with physiologic disturbances to neuronal function accompanying traditional microvascular capillary pathologic disease.

It was in these contexts that the RAGE axis in this newly characterized murine model of NPDR was examined. Not surprisingly, prominent AGE localization within the vitreous cavity was observed. The increased AGE formation in the vitreous cavity of diabetic eyes has been postulated to increase collagen cross-linking and cause vitreous changes characteristic of diabetic eyes, well-recognized phenomena sometimes referred to as diabetic vitreoschisis or vitreopathy (19, 20). An additional finding of this study was prominent AGE accumulation along the vitreoretinal interface, specifically posterior vitreous cortex and the internal limiting membrane (ILM). Similar to the vitreous cavity, the accumulation of AGEs at the vitreoretinal interface may result in structural alterations that promote mechanical traction in this region. Vitrectomy procedures are sometimes performed to remove tractional effects that promote diabetic macular edema. The localization of AGEs along the vitreoretinal interface is consistent with the concept of a structurally altered posterior hyaloid and ILM capable of promoting subclinical vitreomacular disease in early diabetic retinopathy. AGEs may also exert nontractional, receptor-mediated effects via the RAGE axis. In this regard, an intriguing finding of this study is the localization of RAGE primarily to the Müller cells that extend from the ILM to the external limiting membrane of the retina. The anatomically close apposition of an AGE-laden ILM with the RAGE-expressing footplates suggests that a possible physiologic benefit of diabetic vitrectomy is the removal of AGE ligands from the posterior vitreous cortex and ILM, downregulating the proinflammatory RAGE axis in adjacent Müller cells.

The localization of RAGE to Müller cells raises exciting possibilities for novel roles for these cells in the pathogenesis of diabetic retinopathy. The specific RAGE-dependent mechanisms by which AGEs may alter Müller cell structure and function are the subject of future study. These cells are well known to display a varied repertoire of structural and physiologic properties in the retina. The contact of vasoglial neuronal tissue and especially Müller cells with underlying capillaries in the retina suggests a potential pathophysiologic relationship in diabetic retinopathy, once suggested by Ashton in his Bowman lecture and supported by several recent studies (53). In the setting of diabetes, alteration of the glutamate transporter, speculated in part by oxidation; increased expression of GFAP suggestive of reactive gliosis; and striking upregulation of VEGF all have been detected in Müller cells (54). Indeed, in vitro analyses suggested that incubation of cultured Müller cells with AGEs upregulated expression of VEGF (55). Müller cell ischemia induces phosphorylation of extracellular signal-regulated kinase (ERK) MAPKs in these cells (56), again suggesting that a wide array of changes in gene expression may ensue in these cells when perturbed. The possible RAGE-dependence of these phenomena remains to be determined, but the intimate relationship of RAGE-expressing Müller cells with underlying vascular endothelium suggests a potential role for Müller cell RAGE in neurovascular dysfunction.

In addition to the specific localization of RAGE and its AGE ligands in our study, it was observed that AGEs accumulate in the neurosensory retina with associated amplification of cellular RAGE in the setting of hyperglycemia and early diabetic retinopathy. The diversity by which AGEs may form on the amino groups of proteins, lipids, and DNA is reflected in the variety of locations that these products may accumulate during hyperglycemia, including the serum, extracellular matrix (ECM), and intracellular cytoplasm 19). In this regard, it is noteworthy that significantly different AGE levels by fluorescent studies within cellular proteins among the hyperlipidemic and hyperglycemic phenotypes was not detected. Instead, the retinas with the most severe capillary disease had the highest levels of AGEs detected within the ECM, both by fluorescent and ELISA studies. Hyperglycemia was the most important contributor to the development of these AGEs, as HGNL mice also exhibited increased AGE accumulation in the ECM in these studies (though this increase was only significant in this group by ELISA). Consistent with a role for RAGE ligands such as AGEs in the development of retinopathy, significant upregulation of RAGE transcripts was detected in the retinas of HGHL mice that had the highest AGE accumulation and retinal disease. The amplification of RAGE in the setting of its ligands is consistent with the known biology of RAGE in other organ systems, and this property magnifies the effect of the RAGE axis in local cellular responses (26, 40).

Importantly, in this study, antagonism of the RAGE axis ameliorated both neuronal dysfunction and vascular disease. The electrophysiologic benefit that was observed suggests that RAGE contributes to neuronal dysfunction in the diabetic retina. The mechanisms of oscillatory potential generation in the normal retina, the associated alterations observed in these neuronal responses in diabetic eyes, and the extent to which altered Müller cell glutamate metabolism, signaling and gene expression might contribute to perturbation of these signals remains to be determined. Antagonism of RAGE also reduced the progression of vascular lesions of diabetic retinopathy in hyperglycemic, hyperlipidemic mice. This vascular effect may relate to an a priori neuronal benefit to RAGE-expressing Müller cells, but the ample data on AGE toxicity and perturbation to retinal vascular endothelial cells also suggests that antagonism of circulating serum AGEs with soluble RAGE may reduce these perturbations and resultant anatomic disease. The precise neurovascular mechanisms altered with ligand interaction with RAGE in the retina are not yet elucidated, but the amelioration of neurovascular features of diabetic retinopathy observed in this study identifies the RAGE axis as an important therapeutic target in the prevention and treatment of diabetic complications in the retina.

### REFERENCES

1. Frank R N. Diabetic retinopathy. N Engl J Med. 2004; 350:48-58
2. Gardner T W, Antonetti D A, Barber A J, LaNoue K F, Levison S W. Diabetic retinopathy: More than meets the eye. Surv Opthalmol. 2002; 47 Suppl 2:S253-262
3. Lieth E, Gardner T W, Barber A J, Antonetti D A. Retinal neurodegeneration: Early pathology in diabetes. Clin Experiment Opthalmol. 2000; 28:3-8
4. Aiello L P, Avery R L, Arrigg P G, et al. Vascular endothelial growth factor in ocular fluid of patients with diabetic retinopathy and other retinal disorders. N Engl J Med. 1994; 331:1480-1487
5. The Diabetes Control and Complications Trial Research Group. The effect of intensive treatment of diabetes on the development and progression of long-term complications in insulin-dependent diabetes mellitus. N Engl J Med. 1993; 329:977-986
6. The UK Prospective Diabetes Study (UKPDS) Group. Intensive blood-glucose control with sulphonylureas or insulin compared with conventional treatment and risk of complications in patients with type 2 diabetes (UKPDS 33). Lancet. 1998; 352:837-853
7. The UK Prospective Diabetes Study (UKPDS) Group. Tight blood pressure control and risk of macrovascular and microvascular complications in type 2 diabetes: UKPDS 38. BMJ. 1998; 317:703-713
8. Kissebah A H, Kohner E M, Lewis B, Siddiq Y K, Lowy C, Fraser T R. Plasma-lipids and glucose/insulin relationship in non-insulin-requiring diabetics with and without retinopathy. Lancet. 1975; 1:1104-1108
9. Chew E Y, Klein M L, Ferris F L, 3rd, et al. Association of elevated serum lipid levels with retinal hard exudate in diabetic retinopathy. Early Treatment Diabetic Retinopathy Study (ETDRS) Report 22. Arch Opthalmol. 1996; 114:1079-1084
10. Davis M D, Fisher M R, Gangnon R E, et al. Risk factors for high-risk proliferative diabetic retinopathy and severe visual loss: Early Treatment Diabetic Retinopathy Study Report #18. Invest Opthalmol V is Sci. 1998; 39:233-252
11. Sen K, Misra A, Kumar A, Pandey R M. Simvastatin retards progression of retinopathy in diabetic patients with hypercholesterolemia. Diabetes Res Clin Pract. 2002; 56:1-11
12. Gordon B, Chang S, Kavanagh M, et al. The effects of lipid lowering on diabetic retinopathy. Am J Opthalmol. 1991; 112:385-391
13. Fried L F, Forrest K Y, Ellis D, Chang Y, Silvers N, Orchard TJ. Lipid modulation in insulin-dependent diabetes mellitus: Effect on microvascular outcomes. J Diabetes Complications. 2001; 15:113-119
14. Schleicher E D, Wagner E, Nerlich A G. Increased accumulation of the glycoxidation product N(epsilon)-(carboxymethyl)lysine in human tissues in diabetes and aging. J Clin Invest. 1997; 99:457-468
15. Hammes H P, Alt A, Niwa T, et al. Differential accumulation of advanced glycation end products in the course of diabetic retinopathy. Diabetologia. 1999; 42:728-736
16. Lu M, Kuroki M, Amano S, et al. Advanced glycation end products increase retinal vascular endothelial growth factor expression. J Clin Invest. 1998; 101:1219-1224
17. Tolentino M J, Miller J W, Gragoudas E S, et al. Intravitreous injections of vascular endothelial growth factor produce retinal ischemia and microangiopathy in an adult primate. Opthalmology. 1996; 103:1820-1828
18. Moore T C, Moore J E, Kaji Y, et al. The role of advanced glycation end products in retinal microvascular leukostasis. Invest Opthalmol V is Sci. 2003; 44:4457-4464
19. Stitt A W. The role of advanced glycation in the pathogenesis of diabetic retinopathy. Exp Mol Pathol. 2003; 75:95-108
20. Sebag J. Abnormalities of human vitreous structure in diabetes. Graefes Arch Clin Exp Opthalmol. 1993; 231:257-260
21. Kern T S, Engerman R L. Pharmacological inhibition of diabetic retinopathy: Aminoguanidine and aspirin. Diabetes. 2001; 50:1636-1642
22. Gardiner T A, Anderson H R, Stitt A W. Inhibition of advanced glycation end-products protects against retinal capillary basement membrane expansion during long-term diabetes. J Pathol. 2003; 201:328-333
23. Stitt A, Gardiner T A, Alderson N L, et al. The AGE inhibitor pyridoxamine inhibits development of retinopathy in experimental diabetes. Diabetes. 2002; 51:2826-2832
24. Hammes H P, Du X, Edelstein D, et al. Benfotiamine blocks three major pathways of hyperglycemic damage and prevents experimental diabetic retinopathy. Nat Med. 2003; 9:294-299
25. Schmidt A M, Vianna M, Gerlach M, et al. Isolation and characterization of two binding proteins for advanced glycosylation end products from bovine lung which are present on the endothelial cell surface. J Biol Chem. 1992; 267:14987-14997
26. Yan S F, Ramasamy R, Naka Y, Schmidt A M. Glycation, inflammation, and RAGE: a scaffold for the macrovascular complications of diabetes and beyond. Circ Res. 2003; 93:1159-1169.
27. Hofmann M A, Drury S, Fu C, et al. RAGE mediates a novel proinflammatory axis: A central cell surface receptor for s100/calgranulin polypeptides. Cell. 1999; 97:889-901
28. Hori O, Brett J, Slattery T, et al. The receptor for advanced glycation end products (RAGE) is a cellular binding site for amphoterin. Mediation of neurite outgrowth and co-expression of RAGE and amphoterin in the developing nervous system. J Biol Chem. 1995; 270:25752-25761
29. Chavakis T, Bierhaus A, Al-Fakhri N, et al. The pattern recognition receptor (RAGE) is a counterreceptor for leukocyte integrins: A novel pathway for inflammatory cell recruitment. J Exp Med. 2003; 198:1507-1515
30. Schmidt A M, Hori O, Chen J X, et al. Advanced glycation endproducts interacting with their endothelial receptor induce expression of vascular cell adhesion molecule-1 (VCAM-1) in cultured human endothelial cells and in mice. A potential mechanism for the accelerated vasculopathy of diabetes. J Clin Invest. 1995; 96:1395-1403
31. Wautier J L, Zoukourian C, Chappey O, et al. Receptor-mediated endothelial cell dysfunction in diabetic vasculopathy. Soluble receptor for advanced glycation end products blocks hyperpermeability in diabetic rats. J Clin Invest. 1996; 97:238-243
32. Laver N M, Robison W G, Jr., Pfeffer B A. Novel procedures for isolating intact retinal vascular beds from diabetic humans and animal models. Invest Opthalmol V is Sci. 1993; 34:2097-2104
33. Kuwabara T, Cogan DG. Studies of retinal vascular patterns. I. Normal architecture. Arch Opthalmol. 1960; 64:904-911
34. Algvere P. Clinical studies on the oscillatory potentials of the human electroretinogram with special reference to the scotopic b-wave. Acta Opthalmol (Copenh). 1968; 46:993-1024
35. Segawa M, Hirata Y, Fujimori S, Okada K. The development of electroretinogram abnormalities and the possible role of polyol pathway activity in diabetic hyperglycemia and galactosemia. Metabolism. 1988; 37:454-460
36. Park L, Raman K G, Lee K J, et al. Suppression of accelerated diabetic atherosclerosis by the soluble receptor for advanced glycation endproducts. Nat Med. 1998; 4:1025-1031
37. Yin D. Biochemical basis of lipofuscin, ceroid, and age pigment-like fluorophores. Free Radic Biol Med. 1996; 21:871-888
38. Meerwaldt R, Graaff R, Oomen P H, et al. Simple non-invasive assessment of advanced glycation endproduct accumulation. Diabetologia. 2004; 47:1324-1330
39. Livak K J, Schmittgen T D. Analysis of relative gene expression data using real-time quantitative PCR and the 2(-Delta Ddelta C(T)) method. Methods. 2001; 25:402-408
40. Li J, Schmidt A M. Characterization and functional analysis of the promoter of RAGE, the receptor for advanced glycation end products. J Biol Chem. 1997; 272:16498-16506
41. Engerman R L, Kern T S. Retinopathy in animal models of diabetes. Diabetes Metab Rev. 1995; 11:109-120
42. Chua S C, Jr., Chung W K, Wu-Peng X S, et al. Phenotypes of mouse diabetes and rat fatty due to mutations in the OB (leptin) receptor. Science. 1996; 271:994-996
43. Lyons T J, Jenkins A J, Zheng D, et al. Diabetic retinopathy and serum lipoprotein subclasses in the DCCT/EDIC cohort. Invest Opthalmol V is Sci. 2004; 45:910-918
44. Chen W, Jump D B, Grant M B, Esselman W J, Busik J V. Dyslipidemia, but not hyperglycemia, induces inflammatory adhesion molecules in human retinal vascular endothelial cells. Invest Opthalmol V is Sci. 2003; 44:5016-5022
45. Tzekov R, Arden G B. The electroretinogram in diabetic retinopathy. Surv Opthalmol. 1999; 44:53-60
46. Han Y, Bearse M A, Jr., Schneck M E, Barez S, Jacobsen C H, Adams A J. Multifocal electroretinogram delays predict sites of subsequent diabetic retinopathy. Invest Opthalmol Vis Sci. 2004; 45:948-954
47. Bresnick G H, Palta M. Predicting progression to severe proliferative diabetic retinopathy. Arch Opthalmol. 1987; 105:810-814
48. Bresnick G H. Diabetic retinopathy viewed as a neurosensory disorder. Arch Opthalmol. 1986; 104:989-990
49. Barber A J, Lieth E, Khin S A, Antonetti D A, Buchanan AG, Gardner TW. Neural apoptosis in the retina during experimental and human diabetes. Early onset and effect of insulin. J Clin Invest. 1998; 102:783-791
50. Zeng X X, Ng Y K, Ling E A. Neuronal and microglial response in the retina of streptozotocin-induced diabetic rats. Vis Neurosci. 2000; 17:463-471
51. Mizutani M, Gerhardinger C, Lorenzi M. Müller cell changes in human diabetic retinopathy. Diabetes. 1998; 47:445-449
52. Shirao Y, Kawasaki K. Electrical responses from diabetic retina. Prog Retin Eye Res. 1998; 17:59-76
53. Ashton N. Bowman lecture. The blood-retinal barrier and vaso-glial relationships in retinal disease. Trans Opthalmol Soc U K. 1965; 85:199-230
54. Amin R H, Frank R N, Kennedy A, Eliott D, Puklin J E, Abrams G W. Vascular endothelial growth factor is present in glial cells of the retina and optic nerve of human subjects with nonproliferative diabetic retinopathy. Invest Opthalmol Vis Sci. 1997; 38:36-47
55. Hirata C, Nakano K, Nakamura N, et al. Advanced glycation end products induce expression of vascular endothelial growth factor by retinal Müller cells. Biochem Biophys Res Commun. 1997; 236:712-715
56. Roth S, Shaikh A R, Hennelly M M, Li Q, Bindokas V, Graham C E. Mitogen-activated protein kinases and retinal ischemia. Invest Opthalmol Vis Sci. 2003; 44:5383-5395
57. Neeper, M., et al. Coning and expression of RAGE: a cell surface receptor for advanced glycation end products of proteins. J. Biol Chem. 1992; 267: 14998-15004
58. Stern, D., et al., U.S. Pat. No. 6,555,651, issued Apr. 29, 2003′.

Foster City, Calif.) according to manufacturer's instructions. Primers and probes for β-actin and RAGE were designed using Primer Express® software (Applied Biosystems). To confirm specific amplification of the target mRNA, an aliquot of the PCR product was analyzed using gel electrophoresis. The sequences of the primers and probe were as follows: for β-actin, 5′-ACG GCC AGG TCA TCA CTA TTG-3′ (forward) (SEQ ID NO:10), 5′-TGG ATG CCA CAG GAT TCC AT-3′ (reverse) (SEQ ID NO:11) and 5′-6FAM-ACG TCT ACC AGC GAA GCT ACT GCC GTC-TAMRA-3′ (probe) (SEQ ID NO:12); for RAGE, 5′-GGA CCC TTA GCT GGC ACT TAG A-3′ (forward) (SEQ ID NO:13), 5′-GAG TCC CGT CTC AGG GTG TCT-3′ (reverse) (SEQ ID NO:14) and 5′-6FAM-ATT CCC GAT GGC AAA GAA ACA CTC GTG-TAMRA-3′ (probe) (SEQ ID NO:15) (Applied Biosystems). Real-time PCR was conducted using ABI PRISM 7900HT Sequence Detection System and results were analyzed using the 2^{-ΔΔc}T method (39). Experiments were repeated 3 times, and statistical analysis was performed as described below.

1. A method for treating diabetic retinopathy in a subject afflicted therewith, comprising administering to the subject's eyes a therapeutically effective amount of soluble RAGE or a derivative thereof, thereby treating diabetic retinopathy in the subject.

2-20. (canceled)

21. A method for inhibiting the onset of diabetic retinopathy in a subject afflicted therewith, comprising administering a prophylactically effective amount of soluble RAGE or a derivative thereof to the subject's eyes, thereby inhibiting the onset of diabetic retinopathy in the subject.

22-40. (canceled)
